(19) United States
(12) Patent Application Publication
(10) Pub. No.: US 2020/0121237 A1
(43) Pub. Date: Apr. 23, 2020

(54) A METHOD AND SYSTEM FOR MONITORING ATTENTION OF A SUBJECT

(71) Applicant: MINDSEYE DIAGNOSTICS LTD., Givat Haim Ichud (IL)

(72) Inventors: Dov YELLIN, Raanana (IL); Anat BARNEA, Givat Haim Ichud (IL); Eran FERRI, Hof it (IL); Boaz BRILL, Rehovot (IL)

(21) Appl. No.: 16/477,886

(22) PCT Filed: Jan. 17, 2018
(86) PCT No.: PCT/IL2018/050060

§ 371 Date: Jul. 14, 2019

Related U.S. Application Data
(60) Provisional application No. 62/446,849, filed on Jan. 17, 2017.

(51) Int. Cl. A61B 5/16; A61B 5/00; A61B 3/11

(52) U.S. Cl. CPC A61B 5/168; A61B 5/163; A61B 5/4884; A61B 3/112; A61B 5/0077

(57) ABSTRACT

Methods and systems, which are computerized, monitor the attention level of a subject, by obtaining at least one set of biomarkers from a subject during a time period, and, calculate, from asymmetries between the biomarkers of the at least one set of obtained biomarkers, a score of attention of the subject during the time period.

### CROSS REFERENCES TO RELATED APPLICATIONS

This application is related to and claims priority from commonly owned U.S. Provisional Patent Application Ser. No. 62/446,849, entitled: A Method for Diagnosing and Monitoring Attention Deficit via Asymmetry Between the Eye Pupils, filed on Jan. 17, 2017, the disclosure of which is incorporated by reference in its entirety herein.

### FIELD OF THE INVENTION

The invention relates to monitoring of the attention level of people (e.g., subjects) over time and the diagnosis of conditions that lower the ability of people to maintain attention over time.

### BACKGROUND OF THE INVENTION

Attention deficit hyperactivity disorder (ADHD) is a neurological developmental disorder affecting both children and adults. It is manifested by persistent patterns of inattention and/or hyperactivity-impulsivity that interrupts daily life. Individuals with ADHD may also have difficulties with focusing their executive function (i.e. the brain's ability to begin an activity, organize itself and manage tasks) and their working memory.

Despite its prevalence, the current diagnostic criteria for ADHD is debated and is based mostly on its clinical presentation (via explicit behavior). That is, via characterization of inattention, hyperactivity, disruptive impulsivity etc., as observed at school, at work, at home and during the diagnostic session. The Diagnostic and Statistical Manual of Mental Disorders, Fifth Edition, (DSM-5), published by the American Psychiatric Association lays out the criteria to be used by mental health professionals when making a diagnosis of ADHD. It lists specific symptoms in all cognitive domains that have been related with ADHD in numerous studies. However the exact criteria are inconsistent across these studies, despite the fact that the most robust findings are impairment in the ability to sustain attention and efficiently retrieve information from working memory.

In practice, psychiatrists and clinicians typically diagnose ADHD cases by implementing the following lengthy assessment procedure:

1. When pertaining to children, parents and teachers fill up the Vanderbilt ADHD Diagnostic Rating Scale (VADPRS) questionnaire, or the Conners Comprehensive Behavior Rating Scales (CBRS) questionnaire.

2. A physical-clinical evaluation is performed by a medical doctor.

3. Cognitive assessment is accomplished using computerized tests, such as T.O.V.A., CPT or BRC to evaluate cognitive abilities\deficiencies.

4. In rare cases, EEG recordings are performed as well to rule out the possibility of more severe brain impairment.

After completing this lengthy evaluation process, the expert uses the mass of information gathered to make a decision about the prevalence of ADHD. However, the numerous steps of these processes, coupled with the need to carefully integrate its result may involve a subjective perspective, which may skew or otherwise affect the result.

Other research into ADHD and attention analysis over the past five decades has looked at the eye's pupil responses with the level of exerted attention. Accumulating evidence from multiple studies indicates that changes in the state of attention are well reflected in the dilation of the pupil (Laeng B. Sirois S. Gredeback G. (2012). Pupillometry: A window to the preconscious? Perspectives on Psychological Science, 7 (1), 18-27). Thus implying that ongoing measures of pupil diameter may be used as a psychophysiological gauge of mental effort and attention.

The suggested underlying cause for this relation between attention and the pupil was found to lie in a brain-stem nucleus, called the locus coeruleus (LC) which plays a fundamental role in the noradrenaline (NE) system (Sara, S. J., 2009. The locus coeruleus and noradrenergic modulation of cognition. Nat Rev Neurosci 10, 211-223). Additionally, Slamovits T L, Glaser J S, Mbekeani J, in, The Pupils and Accommodation in Neuro-Ophthalmology, (Glaser J S, ed) 4th ed., J B Lippincott, Philadelphia, Pa. (2002) suggested that, as a rule of thumb, “the pupils are round and practically equal in diameter”. Therefore, it has also been widely believed, so far, that the change in the diameter of two pupils of a person's eyes over the course of time is highly symmetric.

### SUMMARY OF THE INVENTION

The present invention is directed to a method for diagnosis and/or monitoring of attention deficit in a subject via one or more biomarkers measured from images of the subject. For example, the images are of the left and right eyes of a subject, including observations of asymmetric behavior of the pupils of the eyes.

The present invention provides methods for diagnosing ADHD and Attention Deficit Disorder (ADD) using a universal biomarker.

The present invention is directed to methods and systems, which are computerized, and which monitor the attention level of a subject, by obtaining at least one set of biomarkers from a subject during a time period, and, calculate, from asymmetries between the biomarkers of the at least one set of obtained biomarkers, a score of attention of the subject during the time period.

The present invention is directed to methods for diagnosing ADHD and ADD using biomarkers derived from the measurement of asymmetries from images of the subject, such as from eye pupils.

The present invention is directed to methods and systems for diagnosing and/or monitoring of ADHD and ADD using an indicator of asymmetry in the pupils of the eyes.

The present invention provides an apparatus that supports the measurement of attention levels in a subject, and, for example, includes a camera.

The present invention provides a shorter and more rigorous process for determining the presence of ADHD, by using a neurobiological biomarker. This enables objective monitoring of attention of the subject for the diagnosis of ADHD. Moreover, the aforementioned biomarkers are using phenomenological markers alone. The present invention provides a method for monitoring attention level of a subject, comprising:
(a) obtaining a series of images containing the face of the subject and specifically containing both eyes of a subject;
(b) measuring a series of biometrics pertaining to facial parameters in said series of images, and specifically to the pupil diameters or pupil areas for each pupil (left and right) from said series of images;
(c) computing a measure of asymmetry based on said biometrics, and specifically a measure of asymmetry between left and right pupils, based on fluctuations in their size with time; and,
(d) Compiling from said measure of asymmetry and other possible parameters a score of attention which could be temporal or general.

Optionally, the score of attention is measured while the subject is engaged in a cognitive task.

Optionally, the score of attention is compared to a predetermined threshold supporting a decision regarding the attention capacity of the subject.

Optionally, the series of images is divided into at least two, optionally partially overlapping sub-series and each sub-series is separately analyzed, obtaining a temporal score of attention.

Optionally, the temporal score of attention is presented to the subject in real time.

Embodiments of the invention are directed to a method for monitoring the attention level of a subject. The method comprises: obtaining at least one set of biomarkers from the left side of the face and the right side of the face of the subject (for example, the face is a symmetric or at least substantially symmetric part of the body) during at least one time period (e.g., a time window); and, calculating, by a processor, from asymmetries between the biomarkers of the at least one set of obtained biomarkers, a score of attention of the subject during the at least one time period.

Optionally, for the aforementioned method, the at least one time period may also be a plurality of time periods and the at least one time window may be a plurality of partially overlapping time windows.

Optionally, the at least one set of biomarkers includes a plurality of sets of biomarkers, and the obtaining the at least one set of biomarkers includes: obtaining, from an imaging apparatus, a plurality of images of the face of the subject over the at least one time period; and, defining the biomarkers for each set of biomarkers from each image of the obtained plurality of images.

Optionally, the imaging apparatus includes at least one of cameras and eye trackers.

Optionally, the obtaining the at least one set of biomarkers is performed by at least one of a camera or an eye tracker.

Optionally, the biomarkers are associated with left and right eyes of the subject.

Optionally, the biomarkers include at least one of pupil diameter or pupil area.

Optionally, the obtaining the at least one set of biomarkers occurs during the performance of a cognitive task.

Optionally, the calculating the score of attention of the subject includes calculating at least one correlation between the biomarkers relating to: 1) the left side of the face over the at least one time period, and, 2) the right side of the face, over the at least one time period.

Optionally, method additionally comprises: obtaining an overall metric of attention of the subject by combining each said score of attention over the at least one time period.

Optionally, the at least one time period includes a plurality of time periods.

Optionally, the overall metric for attention is compared to a threshold in order to diagnose Attention Deficit Disorder (ADD) or Attention Deficit Hyperactivity Disorder (ADHD).

Optionally, the score of attention is presented to the subject in real time.

Optionally, the cognitive task includes presenting to the subject at least one of visual and auditory contents.

Optionally, the presenting the visual contents includes alternating presentations of a set of visual triggers such that no more than one visual trigger is presented at any given time.

Optionally, the auditory contents include at least one of single tones, music or speech.

Embodiments of the invention are directed to a system for monitoring the attention level of a subject. The system comprises: an eye tracker for obtaining at least one set of biomarkers from the left side of the face and the right side of the face of the subject during at least one time period; and, a processor for receiving data associated with the eye tracker. The processor is programmed to: calculate asymmetries between the biomarkers of the at least one set of obtained biomarkers, a score of attention of the subject during the at least one time period.

Optionally, the eye tracker includes an imaging apparatus, and wherein the at least one set of biomarkers includes a plurality of sets of biomarkers, and the processor is additionally programmed to: obtain, from the imaging apparatus, a plurality of images of the face of the subject over the at least one time period; and, define the biomarkers for each set of biomarkers from each image of the obtained plurality of images.

Optionally, the imaging apparatus includes at least one of cameras and eye trackers.

Optionally, the eye tracker for obtaining the at least one set of biomarkers includes at least one of an eye tracking device or a camera.

Optionally, the processor is additionally programmed to associate the biomarkers with left and right eyes of the subject.

Optionally, the biomarkers include at least one of pupil diameter or pupil area.

Optionally, the processor is additionally programmed to calculate the score of attention of the subject by calculating at least one correlation between the biomarkers relating to: 1) the left side of the face over the at least one time period; and, 2) the right side of the face, over the at least one time period.

Optionally, the processor is additionally programmed to obtain an overall metric of attention of the subject by combining each said score of attention over the at least one time period.

Optionally, the processor is additionally programmed to define the at least one time period to include a plurality of time periods.

Optionally, the processor is additionally programmed to compare the overall metric for attention to a threshold in order to diagnose Attention Deficit Disorder (ADD) or Attention Deficit Hyperactivity Disorder (ADHD).

Optionally, the system additionally comprises a display in electrical and/or data communication with the processor, and the processor is additionally programmed to send the score of attention to the display for presentation in real time.

Optionally, the system of additionally comprises at least one of lights, display or speakers for presenting a cognitive task in at least one of visual or auditory content.

Optionally, the lights or the display are activatable to define visual triggers for the cognitive task, and are controllable such that no more than one visual trigger is presented at any given time.

Optionally, the auditory content from the speakers includes at least one of single tones, music or speech.

This document references terms that are used consistently or interchangeably herein. These terms, including variations thereof, are as follows.

A “computer” includes machines, computers and computing or computer systems (for example, physically separate locations or devices), servers, computer and computerized devices, processors, processing systems, computing cores (for example, shared devices), and similar systems, workstations, modules and combinations of the aforementioned. The aforementioned “computer” may be in various types, such as a personal computer (e.g., laptop, desktop, tablet computer), or any type of computing device, including mobile devices that can be readily transported from one location to another location (e smart: phone, personal digital assistant (PDA), mobile telephone or cellular telephone).

A “server” is typically a remote computer or remote computer system, or computer program therein, in accordance with the “computer” defined above, that is accessible over a communications medium, such as a communications network or other computer network, including the Internet. A “server” provides services to, or performs functions for, other computer programs (and their users), in the same or other computers. A server may also include a virtual machine, a software based emulation of a computer.

An “application”, includes executable software, and optionally, any graphical user interfaces (GUI), through which certain functionality may be implemented.

All the above and other characteristics and advantages of the invention will become well understood through the following illustrative and non-limitative description of embodiments thereof, with reference to the appended drawings.

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Attention is now directed to the drawings, where like reference numerals or characters indicate corresponding or like components. In the drawings:

FIG. 1 is a schematically shows a cognitive task requiring the subject to identify a specific geometrical shape, used in a feasibility study of the proposed method;

FIG. 2A is a block diagram of a system in accordance with an embodiment of the invention;

FIG. 2B is a block diagram of the controller of FIG. 2A;

FIG. 2C is a block diagram of a system in accordance with another embodiment of the invention;

FIG. 2D schematically shows the main steps of a method for the calculation of a score of attention from the measurement of pupil sizes;

FIG. 3; schematically show pupil sizes of both eyes from a sample subject over a period of approximately 6 minutes;

FIGS. 4A and 4B schematically show a table of the attention score and a graph of the sliding window correlation for each of the 21 participants of the study, including normal subjects in FIG. 4A and ADHD subjects in FIG. 4B; and,

FIGS. 5A and 5B show the mean synchronized trigger response in the left and right eyes, comparing results between two typical subjects, one normal and one with ADHD.

### DETAILED DESCRIPTION OF EMBODIMENTS OF THE PRESENT INVENTION

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more non-transitory computer readable (storage) medium(s) having computer readable program code embodied thereon.

The inventors have found that subjects (e.g., human subjects) characterized by malfunctioning attention faculty are also inclined to exhibit incoherent changes in their pupil size, such that both eyes' pupil sizes do not follow the same pattern. Accordingly, the present invention provides a method for monitoring the attention level of a subject, which may be used for diagnosing or monitoring of Attention Deficit Disorder (ADD) and Attention Deficit Hyperactivity Disorder (ADHD) which uses an indicator of asymmetry in the body, such as in the face and typically in the pupils of the eyes.

The inventors have found that people with attention deficit disorder often show deviations from behaviors characterized as normal. In people with ADD and ADHD, the left and right pupil sizes often display different patterns over time, both at rest and while the person is attempting to attend to a cognitive task. As all muscle activities, eye muscles, including the pupils, are controlled by the opposite hemisphere of the brain, i.e., right eye muscles are controlled by the left hemisphere and vice versa. Thus, asymmetry between left and right eye parameters, such as pupil size, are possibly an indication for a reduced coherency between the two hemispheres of the brain, and thus a plausible aspect of mental disorders, e.g., ADD and ADHD.

Accordingly, the present invention relates to a method for diagnosing and/or monitoring attention levels of subjects by measuring the asymmetry between left and right biomarkers of the eyes. Such biomarkers may include any combination of the following biomarkers: (a) pupil size (b) time-domain or frequency-domain analysis of pupil sizes, (c) blinking patterns (d) eye movement patterns. For example, the biomarkers, as disclosed herein, may be scored, with the score for a biomarker represented by a single number describing a single feature in a single image or similar digital representation, for example, left pupil diameter.

According to one aspect of the present invention, measuring of the biomarkers of the eye is done while the subject is attempting to attend to a cognitive task.

The cognitive task could be, for example comprised of a series of cognitive triggers creating a cognitive load. Cognitive triggers may be either visual, auditory or any other sensory inputs or combination thereof. Triggers may specifically stimulate user to perform a predefined cognitive task, for example identifying objects, counting objects, comparing different objects, making decisions, memorizing data, performing mathematical computations, and the like. The subject may be required to respond to each trigger or provide a certain response following several triggers. Triggers may be presented to the user in a periodic manner, with roughly equal time lags between triggers, on in a non-periodic manner Triggers may present equal levels of challenge or different levels of challenge.

The cognitive task may have an overall uniform cognitive load level, for example, by presenting triggers of equal challenge in a periodical manner, or, alternatively, present a non-uniform cognitive load to the subject, such as, for example, an escalating cognitive load, obtained e.g. by gradually increasing the cognitive challenge level presented by each trigger, or e.g. by gradually reducing the time lag between successive triggers. Alternatively, the cognitive task may include reference periods in which eye biomarkers data is registered. However no triggers are presented to the user over a time of, for example, more than 15 seconds, such as more than 30 seconds, and in some cases over 60 seconds. Such reference periods may be placed in the beginning of the cognitive task, at the end of the cognitive task or during the cognitive task. Comparison between reference periods and cognitive task periods may provide additional metrics enabling the differentiation between different types or levels of attention capacity.

Visual triggers may include e.g. different objects, in an object recognition task, as discussed below. Visual triggers may, for example, be alternating presentations of a set of visual triggers such that no more than one visual trigger is presented at any given time.

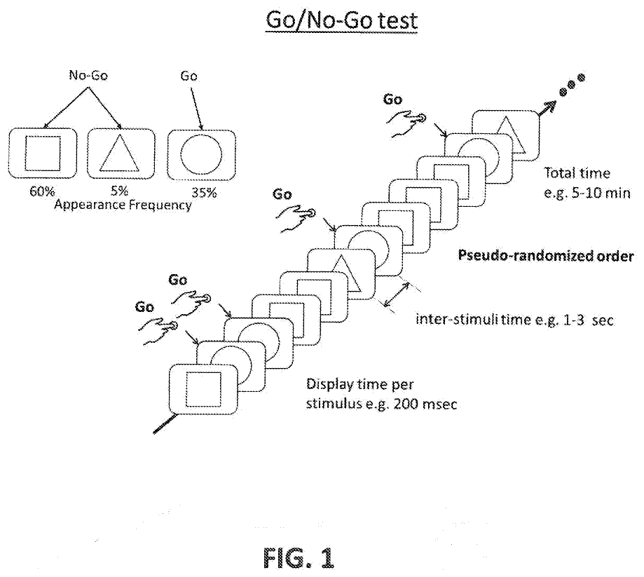

Likewise, auditory triggers may include different types of sounds, as e.g. separate words, meaningful combinations of words such as speech, different natural sounds, tones of different volume or pitch, or sequences of tones such as musical pieces, that can be used in a sound recognition task. Auditory triggers could also be used as distraction while the cognitive load which requires the subject's attention is visual, or vice versa. Alternatively, cognitive load may be produced using any gaming application, any third-party application which is running on the same system which runs the test or on an adjunct system. Alternatively, cognitive load may be produced by exposing the subject to any sensory input of sufficient information content, for example, requiring the subject to read a sufficiently long text, having the subject view a video clip which requires some cognitive effort to understand, and the like. An example of a cognitive task based on visual inputs is shown in FIG. 1 and will be described below.

According to another aspect of the present invention, eye biomarkers are measured without presenting a cognitive task to the subject, e.g., deliberately allowing the subject to enter a state of rest and mind wandering, for example, by letting the subject focus on a dot in the center of an empty screen. According to yet another aspect of this invention, biomarker results from the resting period are used in combination with biomarker results obtained during a cognitive task in order to improve the results of the overall attention assessment process.

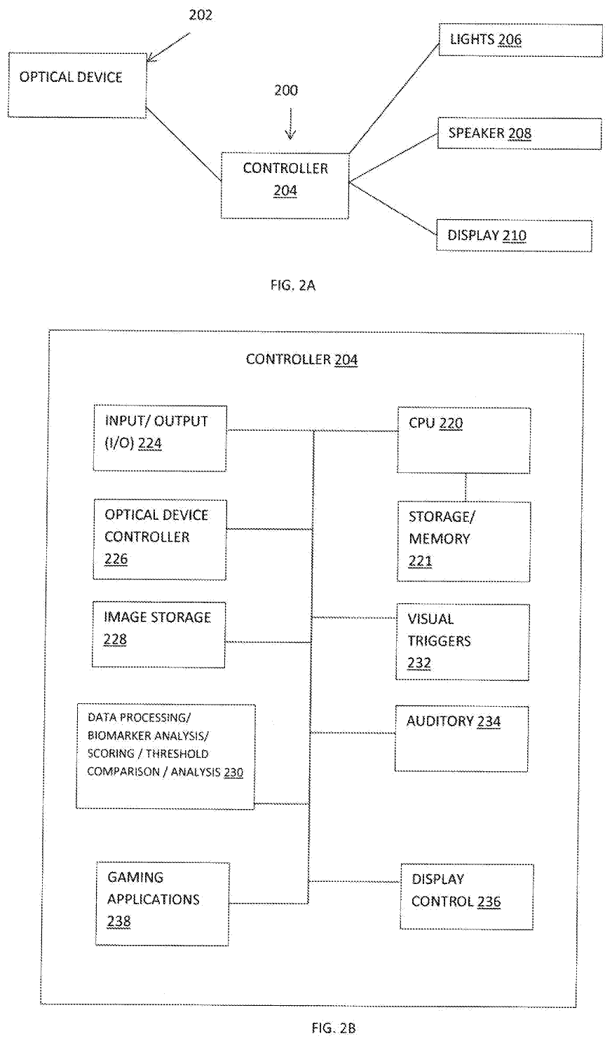

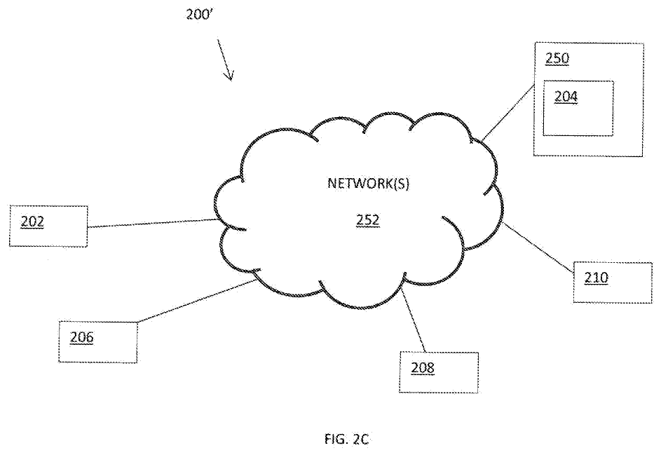

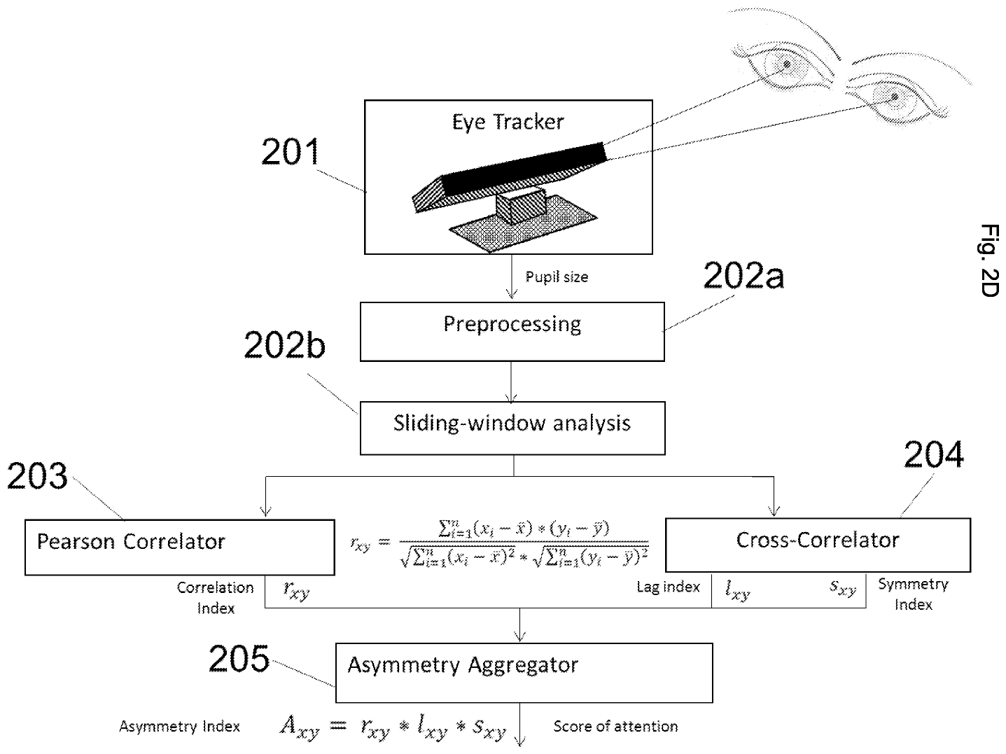

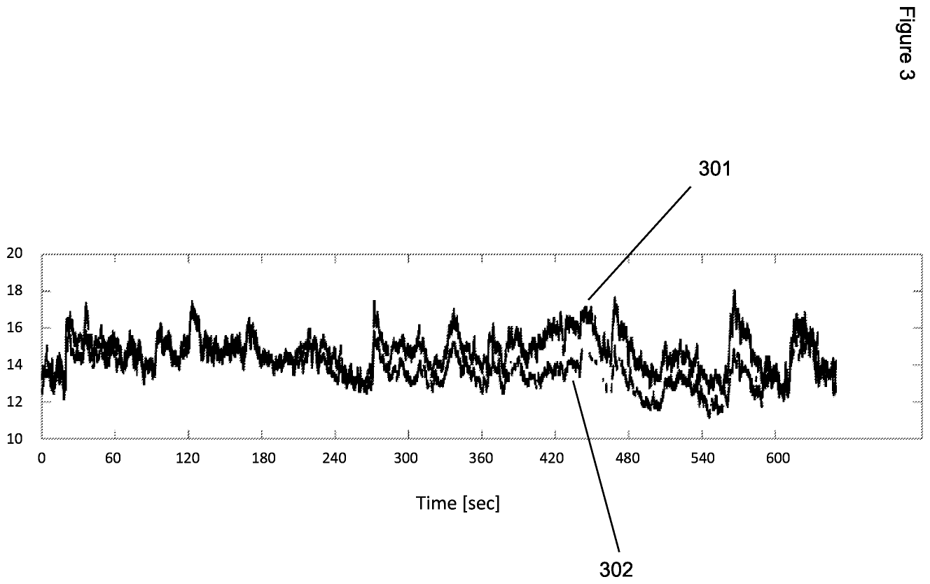

FIG. 2A shows a diagram of an exemplary system 200 used in performing the invention. The system 200 includes an optical device 202, for obtaining the requisite biomarkers, linked to a controller 204, which is in turn linked to lights 206, one or more speakers 208 and a display 210, viewable by the subject being analyzed. “Linked” as used herein includes both wired or wireless links, either direct or indirect, such that the computers, including, servers, components and the like, are in electronic and/or data communications with each other.

The optical device 202, which obtains the biomarkers, includes, for example, an imaging apparatus, such as a camera or eye tracker, both, for example, with image processing capabilities, and eye tracking glasses.

The lights 204 are optional, and are a series of lights to provide visual triggers, as detailed herein. The lights 204 are also used, for example, to illuminate the face of the subject. The lights 204 may also be a light-emitting display. The brightness of the light source, and hence, the lights, is automatically adjusted in order to provide sufficient illumination to the face of the subject, as is measurable by the spatial noise in the image.

The speakers 208, or auditory outputs, provide auditory contents such as single tones, music and speech, at various intervals. The display 210 provides both a means to display different visual triggers that are part of the cognitive load, as e.g., video, geometric shapes and the like, and is also optionally used to provide audio and visual indications of a score and/or diagnosis to the subject, for example, in real time. The speakers 208 may also be, for example, loudspeakers or headphones. The output from the speakers 208 serves as auditory inputs to the subject during the measurement, for example, auditory triggers, synchronized or not with visual triggers, background noise, such as white noise, or music.

FIG. 2B shows the controller 204 in detail. The controller 204 is, for example, processor based, and includes a central processing unit (CPU) 220 with associated storage/memory 221, and modules including stored machine executable instructions to be executed by the CPU 220, the modules including those for inputs and outputs (I/O) 224, optical device control 226, image storage 228, data processing/biomarker analysis/scoring/threshold comparison and analysis 230, visual triggers 232, auditory 234, display control 236 and gaming applications 238.

The Central Processing Unit (CPU) 220 is formed of one or more processors, including microprocessors, and are programmed to perform the functions and operations detailed herein, including controlling the modules for inputs and outputs (I/O) 224, optical device control 226, image storage 228, data processing/biomarker analysis/scoring/threshold comparison and analysis 230, visual triggers 232, audio stimulation 234, display control 236 and gaming applications 238, along with the processes and subprocesses shown in FIG. 2D, as detailed below. The processors are, for example, conventional processors, such as those used in servers, computers, and other computerized devices. For example, the processors may include x86 Processors from AMD and Intel, Xenon® and Pentium® processors from Intel, as well as any combinations thereof.

The storage/memory 221 is any conventional storage media. The storage/memory 221 stores machine executable instructions for execution by the CPU 220, to perform the processes of the invention. The storage/memory 221 also, for example, stores rules and policies, as applied by the CPU 220, for the processes of the invention, as detailed herein. The processors of the CPU 220 and the storage/memory 221, although shown as a single component for representative purposes, may be multiple components.

The Input/Output (I/O) module 224 includes instructions for receiving input, e.g., data from the optical device, and sends output, e.g., signals to the lights 206, speakers 208 and display 210, to perform various actions (detailed herein), based on instructions from the respective visual triggers 232, auditory 236 and display control 236 modules, as processed by the CPU 220.

The optical device control module 226 includes instructions for processing by the CPU 220 to control the optical devices 202, for obtaining the biomarkers. The image storage module 228 stores various images obtained from the optical devices, and is, for example, a storage media.

The data processing/biomarker analysis/scoring/threshold comparison and analysis module 230 provides instructions to the CPU 220 for processing the data associated with biomarkers and sets of biomarkers to determine attention scores (scores of attention), as well as comparing the threshold scores, for determining metrics such as ADD and/or ADHD.

As used herein, a Score of Attention (attention score) is measured over a time window (TW) of, for example, overlapping time windows of, for example, 10-30 seconds, reflecting the attention at a given “point in time”. This is the basic unit of measurement but it is still obtained from multiple images (hundreds). This score is also usable for online monitoring or e.g. for biofeedback if presented to the user in real time. Alternately, each time window interval has a length of e.g., 10-120 seconds, or alternately 20-60 seconds. The time windows are discussed in further detail below.

As used herein, the Overall Metric of Attention is a series of attention scores combined over a longer period of time, e.g. over the time of a cognitive task that is 5 minutes long. This figure is usable, for example, for daily monitoring by the subject or for initial diagnosis by a doctor or other professional or clinician.

The game application module 238 stores various games, which may be executed by the optical device 204 or peripheral devices associated therewith, such as headsets, e.g., Augmented Reality and Virtual Reality Headsets, displays and the like (not shown).

FIG. 2C shows a system 200′ similar to the system 200, except that the controller 204 is part of a server, 250 linked to a network 252, with the server 250 in the “cloud”. The optical device 202, lights 206, speaker 208 and display are also linked to the network 252. The network 252 includes, for example, public networks such as the Internet and may include single or multiple networks, including data networks and cellular networks. In another embodiment, the system 200 may be embodied on a computer device, such as a smartphone.

FIG. 2D is a schematic flow chart of a method according to one embodiment of the present invention. The first step 261 consists of measuring any biomarker of both eyes of the subject, e.g., the size of both pupils of the subject, using an optical device 202 or instrument, e.g., standard eye-tracking device (such as IR remote eye trackers or eye-tracking glasses), or any apparatus comprising a camera, such as e.g. a smartphone, and recording both eyes' pupil size over a period of time. Recording time may be a predetermined period of time or until sufficiently data has been obtained. Without loss of generally, in the following reference is made to pupil size as the biomarker of choice, however the same methods may be similarly applied for other biomarkers of the eye, as mentioned above or other biomarkers of the face, as e.g. eyebrows positions, mouth corners positions, blinks and the like.

The data received from this step 261, consists of two vectors of numbers, which represent the size of the pupils, e.g. pupil diameter in millimeters (or equivalent index), or pupil area in millimeter squared, as a function of time. The first vector (x-dimension) delineates the pupil size of the left eye over time and the second vector (y-dimension) delineates the size of the right pupil over time.

The second step 262 involves processing of the data received from the first step 261, i.e., the two pupil size vectors. The first sub-step 262a, involves preprocessing the raw pupil-size time-course to deal with temporary loss of signal or noise that may be due to blinks or device artefacts. A corrected vector per each pupil is thereby generated using standard smoothing and interpolation techniques. In a later sub-step 262b the corrected vectors are divided into sliding time-windows. That is, the pupil-size time-course vector of each pupil is broken down into shorter consecutive time-window intervals (TW) of s seconds (where s is a configurable argument having a typical length of 20-120 seconds), with a time shift of d seconds between the start time of each consecutive window (where d is also configurable with typical setting of 1-5 seconds).

In the third step 263, the correlation between aligned TW intervals of both pupils is computed, e.g. using the Pearson correlation coefficient given by the following formula:

$${r}_{xy} = \frac{{\sum }_{i = 1}^{n} ( {x}_{i} - \overline{x} ) * ( {y}_{i} - \overline{y} )}{\sqrt{{\sum }_{i = 1}^{n} {( {x}_{i} - \overline{x} )}^{2}} * \sqrt{{\sum }_{i = 1}^{n} {( {y}_{i} - \overline{y} )}^{2}}}$$

Where xᵢ and yᵢ are the momentary pupil size (at time i) and the terms

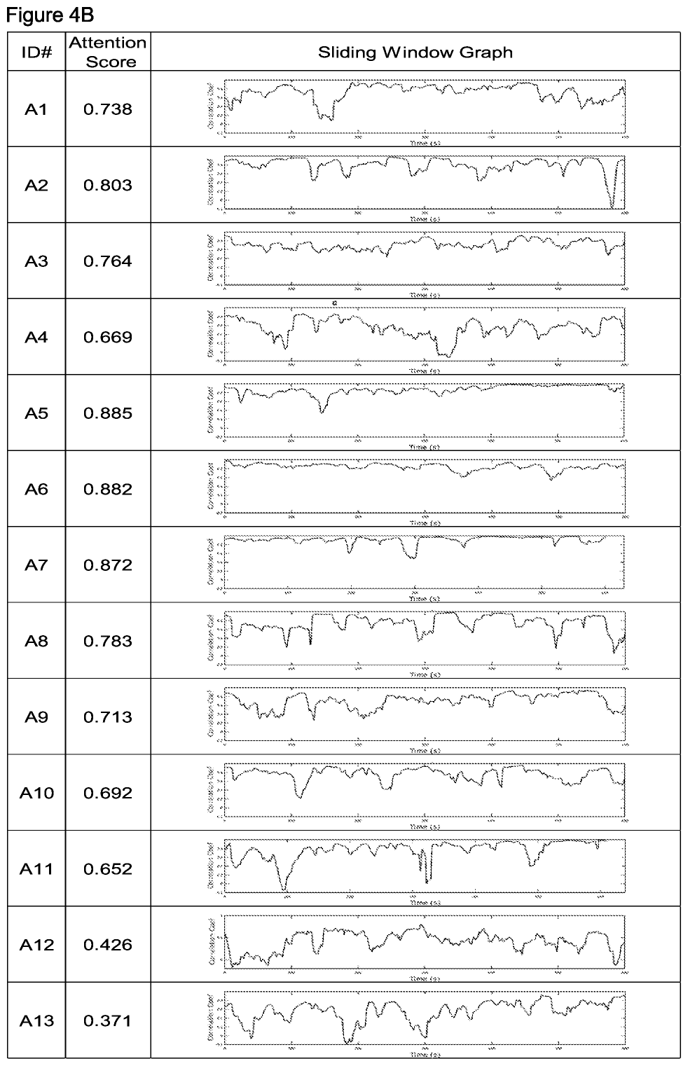

x̅ and y̅ stand for average size of the left and right pupil, respectively, during the time window (TW). Summation is performed across the time-points of the TW, ranging between 1 to n. The possible values for the r_{xy} coefficient in the above formula may fall between −1 to 1, however, under actual “real life” conditions scenarios, expected values are typically greater than 0. Based on our preliminary findings (see below discussion and FIGS. 4A, 4B), results for this coefficient occurring at a level of −0.9 or higher are typical in most normal subjects during a period of good attention, whereas lower values may indicate temporary lack of attention. On the other hand, repeating episodes of lower values are typical of subjects with ADHD and may indicate an attention abnormality.

In step 264, an additional, optional, quantitative analysis is performed, consisting of the cross-correlation between the same TW intervals vectors. This analysis which relies on a time-shifted application of the same Pearson correlation formula as in step 263, provides indication of the lag time to peak correlation between the movements of both pupils, providing additional properties of their asymmetry.

From this cross-correlation analysis step two additional scores may be obtained: (a) a lag index—1_{xy}, normalized between 0-1, where 1 implies expected peak correlation at 0-lag, and 0 denotes abnormal result of lag, e.g. equal or greater than 1 second. (b) a symmetry index—s_{xy}, normalized in the range of 0-1, where 1 indicates perfect minor symmetry for correlation values at corresponding positive and negative lags, and 0 implies a strongly asymmetric behavior, such as an accumulated distance equal to twice or more standard deviation units of the mean across the time-courses of x and y.

Finally, in step 265, a joint asymmetry index is computed through a combination of the three scores—the correlation coefficient—r_{xy}, the lag index—1_{xy} and the symmetry index—s_{xy}. In general, the joint asymmetry index could be any function of r_{xy}, 1_{xy} and s_{xy}, for example a simple multiplication, i.e.:

A_{xy}=r_{xy}*l_{xy}*s_{xy} Equation 1

Alternative, simpler to compute, asymmetry indexes that could also be used include only the correlation r_{xy} for the final index, as in:

A_{xy}=r_{xy} Equation 2

or, using only two of these factors for the final index, as in:

A_{xy}=r_{xy}*1_{xy} Equation 3

A_{xy}=r_{xy}*s_{xy} Equation 4

In the following, A_{xy} will refer in general to any vector of asymmetry index over time computed using any of the above formulae or any other means of computing a measure of asymmetry.

The result A_{xy} is, for example, a vector of scores of attention, providing a temporal indication for the attention of the tested subject, over the time of the test. In the rest of the text this vector is also referred to as “sliding window graph”. One or more overall scores of attention are computed from said vector of measure of attention over time, A_{xy}.

One or more overall attention scores can finally be obtained for the whole test, e.g. by taking the average of the attention scores vector over the entire time-course of the test, or e.g. by using the median value or any other percentile, or, for example, by measuring the variability of the scores over time. Alternatively, correlation between data measured from both eyes using the whole data set can be calculated, without going through the steps of dividing the data into time windows and averaging multiple temporal correlation values. Also alternatively, cross correlation between the eyes data can be computed for different time lags between the eyes and the maximal value can then be chosen as the overall score. The calculating the score of attention of the subject, for example, includes calculating at least one correlation between the biomarkers relating to: 1) the left side of the face over the at least one time period, and, 2) the right side of the face, over the at least one time period.

The processes of blocks 262a, 262b, 263, 264 and 265, are, for example, performed by the module 230 and the CPU 220 in the controller 204 of FIGS. 2A, 2B and 2C.

Another feature includes analyzing the evolution of the temporal score of attention over the course of the cognitive task. For example, comparing the average attention score during a first, earlier part of the cognitive task to the average attention score during a second, later part of the cognitive task, one can determine the general trend over the time of the cognitive task. A general trend indicating a decline in attention score over the time of the cognitive task can be expected for subjects with ADHD who are having a difficulty to maintain high attention level over a prolonged period of time, and could thus be factored into the overall score to reduce the final overall score. Conversely, a general trend indicating an increase in attention score over the time of the cognitive task, could be indicatory e.g. of an initial lack of attention due to other factors, e.g. anxiety resulting from taking the test, which is not related to ADHD, and could thus be factored in to increase the overall attention score. Thus, two subjects having a similar average score, averaging over the whole time of the test, may eventually receive a different overall score based also on the general trend during the time of the test.

The overall attention score obtained using the general method provided above, in any of its variants, can e.g. be used to diagnose attention deficiencies, including ADHD, for example, by comparing the one or more scores obtained by the tested subject to predetermined threshold values. Such values should be derived from statistically significant clinical studies and could depend on the personal parameters of the subject, such as age and sex.

In a monitoring mode of operation, changes in overall attention score(s), or a history of such scores, can be monitored over time in order to gauge the effect of certain activities or actions on the attention level of the tested subject. These activities include, for example, performing physical exercise before or during the test, eating, relaxing or taking any kind of prescribed medication.

According to another aspect of the present invention, the temporal score of attention is presented to the subject in real time. For example, using a smartphone, the triggers for the cognitive task could be presented on the smartphone screen while the smartphone's front camera could capture the subjects pupil image allowing computation of the score of attention in real time. The result is displayed in real time on the smartphone screen, allowing the user to be aware of his or her temporal attention level. Real time display of attention levels may be performed by multiple methods. These methods include, for example, displaying a number, by using a color code, by sound, or by vibration. For example, a color code may use blue color for good (or high) attention and red color for poor or low attention. For example, a full continuous spectrum of colors can be used, e.g. using part of the natural spectrum of the rainbow or a discrete set of colors For example, using sound for displaying results may include modifying the volume or pitch of a tone, or controlling the parameters, e.g. the volume, of a musical piece running throughout the test. For example, using vibration can be done by operating the vibrator whenever attention level is dropping below a certain threshold level or is dropping at a fast rate above a threshold absolute change rate.

According to another embodiment of the invention, the level of the cognitive task presented to the subject is changed by the system, such as systems 200 and 200′ (detailed above) in real time, for example, in a pre-programmed way, or adjusted in response to the measured attention level. Adjustment may be performed, in order to improve measurement accuracy, by exposing the subject to different cognitive task levels, such that the system can better differentiate between similar but non-identical overall attention capacity levels. Adjustments can alternatively be done with the aim of allowing the subject to attempt to improve his or her score during the test, in addition or instead of attempting to provide an overall score by the end of the test.

In another embodiment of the invention, the steps of computing an asymmetry measure of the subject comprise the following steps: defining a set of consecutive images, contained in a pre-determined time window, or pertaining to a certain stage in the cognitive test; identifying and calculating in each image one or more matching pairs of facial parameters in both left and right parts of the face, pupil positions, pupil sizes, eyelid positions (blinks), eyebrow positions, mouth edge positions, etc.; computing a correlation coefficient between the set of facial parameters obtained from the left part of the face and the matching set of facial parameters obtained from the right part of the face.

In another embodiment of the invention, the systems 200, 200′ include steps of computing an asymmetry measure between the two pupils and extracting from the computed asymmetry a score of attention. The method comprises steps including: obtaining two time-matched vectors of pupil sizes of both eyes over time; dividing said vectors into shorter sliding window intervals; computing for each interval the correlation coefficient between right eye and left eye pupil size vector, r_{xy} and interpreting the calculated correlation coefficient as a temporal measure of attention, A_{xy}, from Equations 1-4.

In another embodiment of the present invention, time-matched vectors of pupil sizes of both eyes over time are further analyzed using cross-correlation, adding variable time shifts between left and right vectors and resulting in a lag index, L_{xy} define as the peak correlation found over all time shifts, and a measure of attention over time, A_{xy}, is computed as a product of the indexes:

A_{xy}=r_{xy}*L_{xy}

According to another aspect of the present invention, the method of computing attention score over time from a time series of pupil sizes involved computing the mean value or the median value of said vector of measure of attention over time.

According to another aspect of the present invention, the method of computing attention score over time from a time series of pupil sizes comprises a step of preprocessing, which provides smooth pupil size vectors from raw data, utilizing smoothing and interpolation techniques.

In an embodiment of the present invention, a series of images is obtained using an apparatus which includes an optical device 202 camera and a display, as, for example, a mobile device (e.g., a smartphone), utilizing the display in order to present visual contents to the subject while capturing a series of images by the camera, for example, from front camera of the mobile device. The visual contents may include, a cognitive test including variable geometric shapes, a game including visual aspects or any video film not necessarily including any deliberate cognitive challenges.

While the methods and systems detailed above are shown for monitoring, analyzing and evaluating ADHD, they are also usable for monitoring, analyzing and evaluating ADD.

### EXAMPLE

In order to demonstrate the feasibility of the proposed methods, a feasibility study was conducted with including 21 human subjects. Study subjects were divided into a normal control group, including 8 subjects who did not have any history or any symptoms resembling ADHD, and a positive ADHD group, including 13 subjects that had been previously diagnosed with ADHD or showed clear symptoms of ADHD.

During the study, the 21 subjects were exposed to a cognitive load, while pupil sizes were collected using a standard eye tracker (ET). The cognitive load selected for this study required the subjects to focus for 5-10 minutes on a dot at the center of screen (of the eye tracker), on which 3 optional geometric shapes were been flashed (flash time ˜200 msec) every 1 and 3 seconds, as the subjects participated in a GO/No-GO test, as shown in FIG. 1.

Of the 3 geometric shapes one (square) had a 60% appearance frequency, one (circle) had a 35% appearance frequency and the third shape (triangle), a diverter, had a much lower appearance frequency of 5%. The subject was required to respond by clicking (“Go” condition) a button every time a circle appeared while avoiding to respond (“No-Go” condition) to the other shapes. This GO/NO-GO test generally resembles the “Test of Variables of Attention”, described in Leark et al. (Leark, Greenberg, Kindschi, Dupuy, & Hughes), in, Test of Variables of Attention: Professional Manual. Los Alamitos: The TOVA Company (2007). Task performance parameters were collected but were not a mandatory part of the analysis. As mentioned above, other ways of creating a cognitive load could be used and the specific details of the task used during this study are only given by way of example and do not limit the scope of the invention.

During this study, pupil sizes were recorded using an SMI RedN remote eye-tracker (SensoMotoric Instruments), set at 250 Hz. Subjects sat about 70 cm from a 21″ monitor (display or display screen).

In a separate study, a smartphone camera was successfully utilized for collecting video images from which pupil sizes were extracted and similar results were obtained. Hence, the specifics of the apparatus by which pupil sizes are been measured, including equipment parameters such as frame rate and resolution, are not an essential part of the method, since pupil sizes can be sufficiently accurately determined as a function of time.

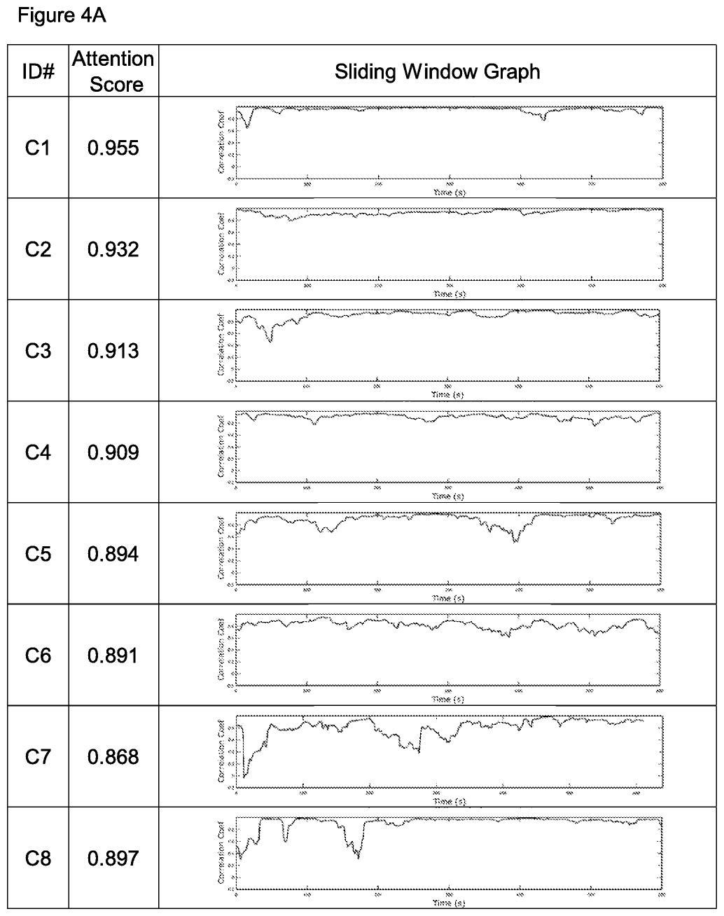

Typical results of one sample subject are provided in FIG. 3, showing the pupil area of the left eye 301 and the right eye 302 over a period of approximately 6 minutes, during which the subject performed a cognitive task. As can be seen, the two curves are highly correlated, practically overlapping. In the beginning of the task, this high correlation exemplifies a high level of attention. However, the correlation is lower in the second half of the task, exemplifying lower attention. In general, it was observed that normal (i.e., those not showing indications and/or scores indicative of ADHD) subjects typically present high correlation between the eyes throughout the task, while diagnosed ADHD subjects typically present longer periods of low correlation between the eyes. The correlation levels can be analyzed using one or more of the methods provided above to provide a measure of correlation between the eyes as a function of time. These correlation graphs can then be summarized using one of the methods described above, to provide an attention level.

In analyzing the data from the 21 subjects of the study, Equation 1 (above) was used to compute a temporal attention score over sliding time windows of 30 seconds each. The mean attention score over the full 10 minute duration of the task to compute an overall attention score per subject was then computed. The results are summarized in FIG. 4A, showing the results of 8 normal subjects, and FIG. 4B, showing the results of 13 ADHD subjects.

As can be appreciated from FIGS. 4A and 4B, different subjects present drastically different attention patterns over time and are rich with information. For example, subjects C1 and C2 show a high and stable attention levels throughout the test and are a good example for people with high attention. On the contrary, subjects A8-A13 show highly unstable levels of attention and even their highest temporary attention levels are often far from being close to 100%. Thus, these subjects exemplify the performance of people with severe ADHD in our test. The large difference in all the characteristics of the graphs shows the strength of the method of the invention and its ability to clearly differentiate between people of different attention levels. This difference is also summarized in the overall attention score which is ˜0.94 for subjects C1 and C2 and is lower than 0.8 for the subjects A8-A13. As was be expected, people are never made up of only two discrete groups and a gray area, including people with various degrees of attention deficits, exists in-between the two extremes. According to this study, subjects that may be regarded as having a mild level of attention deficit, may include A5, A6, A7, C7 and C8. According to medical practice, usually a binary Yes/No decision has to be made, determining if a subject is having a certain condition or not. Based on the finding of this study we could use a threshold of e.g. 0.88 to separate between ADHD subjects and no-ADHD subjects. Using this value, 11 of the 13 subjects were potentially diagnosed as in the ADHD group, indicating a sensitivity of ˜85% and correctly negatively diagnose 7 of the 8 subjects in the control group, indicating a specificity of ˜87%. These results may be further improved using enhanced algorithms, such as those indicated above. In summary, although this study was not a rigorous double-blinded study, it has demonstrated the feasibility and the potential value of the method of the invention.

The aforementioned analysis ignored the timing of the triggers provided to the subject as part of the cognitive task, here, for example,—the flashing times of the different shapes. An alternative way of analyzing the pupils' size over time is by relating the response to the time since the last trigger, known as. time locking. Time locking of pupil responses to visual stimuli events in the abovementioned study, enabled computation of the mean pupil responses of each of the eyes, averaging over all stimuli.

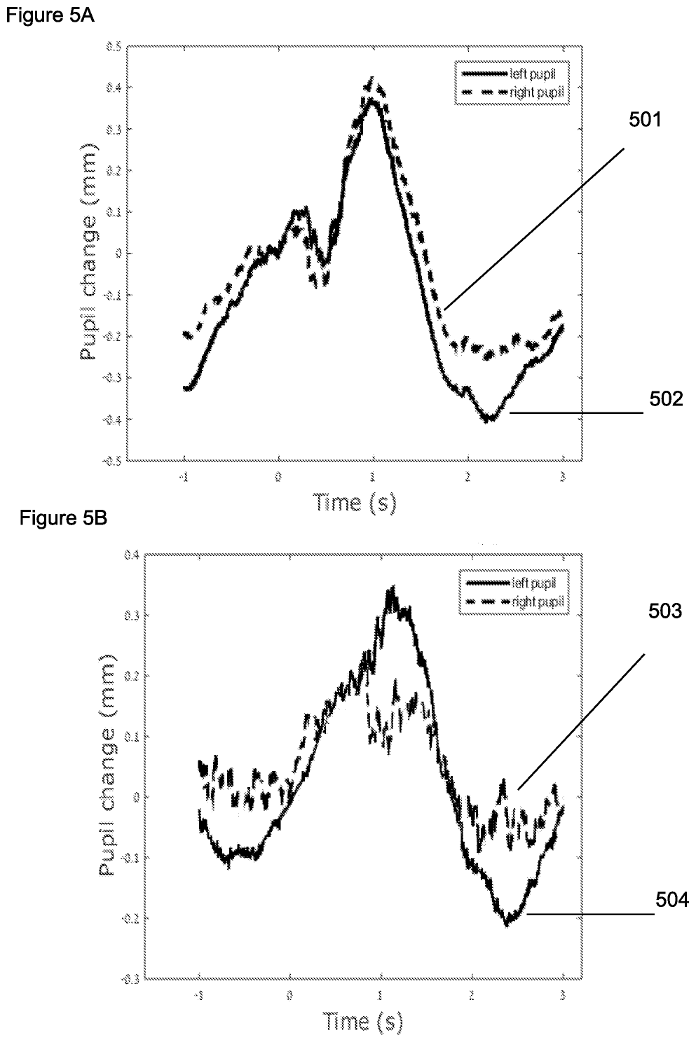

FIGS. 5A and 5B demonstrate the profile of this mean response in the left and right eyes, comparing results between a typical normal subject and a typical ADHD subject. The canonical pupil response pattern peaking at ˜1 s after stimuli onset is clearly visible in both subjects. Results for the right (501, dashed line) and left (502, solid line) pupils of the normal control subject (FIG. 5A) demonstrate highly symmetric responses in both pupils. On the other hand, results for an ADHD subject (FIG. 5B) demonstrate clear incoherence between the two pupils 503 and 504. While the left pupil 504 appears to follow the typical response profile, the right pupil 503 manifests early average constriction in this subject. This result clearly demonstrates how the coherence between the two pupils during a demanding cognitive task may be different between control and ADHD subject. Accordingly, it yet another embodiment of the present invention to compute a measure of attention of a subject using the following steps:

(a) Measure the pupil sizes of both eyes of the subject during a cognitive task comprising multiple cognitive triggers;

(b) Compute the average time-locked pupil sizes of both eyes of the subject;

(c) Compute a measure of asymmetry between the eyes, e.g. by computing correlation.

In another embodiment of the invention, a pupil asymmetry biomarker, in any of the implementations described above, is combined with additional biomarkers, including, for example, blinking frequency, and eye movement parameters, as per the Index of Cognitive Activity (ICA) (Marshall S. P., Aviation, Space, and Environmental Medicine, Vol. 78, No. 5, Section II (May 2007)).

In another embodiment of the invention, an auxiliary optical instrument is used in conjunction with a smartphone (e.g., the auxiliary instrument is mounted to the smartphone) to obtain a series of images. These images are later used for the analysis according to any of the methods described above. For example, the auxiliary optical instrument contains at least one reflective surface, at least two reflective surfaces, or at least one diffusive element, enabling the instrument to illuminate the eyes of the subject, using light emanating from at least one light source, and for example, directing the image of the user's eyes toward the smart phone's rear camera. The light and light source is part of the smartphone. Alternately, the auxiliary optical instrument is electronically connected to the smartphone and comprises at least one light source, optionally operating the infrared (IR) band of the spectrum, and an optional camera.

In another embodiment of the invention, a different task involving a behavioral paradigm other than the Go/No-Go performance test is implemented. This test is used to display the emergence of pupil asymmetry during periods of inattention.

In another embodiment of the invention, a normalized level of pupil symmetry (i.e., reduced asymmetry) is demonstrated in ADHD subjects after standard consumption of alternative ADHD stimulant medication (such as Concerta), or alternately after consumption of coffee (or Caffeine in different forms).

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, non-transitory storage media such as a magnetic hard-disk and/or removable media, for storing instructions and/or data. Optionally, a network connection is provided as well. A display and/or a user input device such as a keyboard or mouse are optionally provided as well.

For example, any combination of one or more non-transitory computer readable (storage) medium(s) may be utilized in accordance with the above-listed embodiments of the present invention. The non-transitory computer readable (storage) medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

As will be understood with reference to the paragraphs and the referenced drawings, provided above, various embodiments of computer-implemented Methods are provided herein, some of which can be performed by various embodiments of apparatuses and systems described herein and some of which can be performed according to instructions stored in non-transitory computer-readable storage media described herein. Still, some embodiments of computer-implemented methods provided herein can be performed by other apparatuses or systems and can be performed according to instructions stored in computer-readable storage media other than that described herein, as will become apparent to those having skill in the art with reference to the embodiments described herein. Any reference to systems and computer-readable storage media with respect to the following computer-implemented methods is provided for explanatory purposes, and is not intended to limit any of such systems and any of such non-transitory computer-readable storage media with regard to embodiments of computer-implemented methods described above. Likewise, any reference to the following computer-implemented methods with respect to systems and computer-readable storage media is provided for explanatory purposes, and is not intended to limit any of such computer-implemented methods disclosed herein.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

The above-described processes including portions thereof can be performed by software, hardware and combinations thereof. These processes and portions thereof can be performed by computers, computer-type devices, workstations, processors, micro-processors, other electronic searching tools and memory and other non-transitory storage-type devices associated therewith. The processes and portions thereof can also be embodied in programmable non-transitory storage media, for example, compact discs (CDs) or other discs including magnetic, optical, etc., readable by a machine or the like, or other computer usable storage media, including magnetic, optical, or semiconductor storage, or other source of electronic signals.

The processes (methods) and systems, including components thereof, herein have been described with exemplary reference to specific hardware and software. The processes (methods) have been described as exemplary, whereby specific steps and their order can be omitted and/or changed by persons of ordinary skill in the art to reduce these embodiments to practice without undue experimentation. The processes (methods) and systems have been described in a manner sufficient to enable persons of ordinary skill in the art to readily adapt other hardware and software as may be needed to reduce any of the embodiments to practice without undue experimentation and using conventional techniques.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for monitoring the attention level of a subject, comprising:
obtaining at least one set of biomarkers from the left side of the face and the right side of the face of the subject during at least one time period; and,
calculating, by a processor, from asymmetries between the biomarkers of the at least one set of obtained biomarkers, a score of attention of the subject during the at least one time period.

2. The method of claim 1, wherein the at least one set of biomarkers includes a plurality of sets of biomarkers, and the obtaining the at least one set of biomarkers includes:
obtaining, from an imaging apparatus, a plurality of images of the face of the subject over the at least one time period; and,
defining the biomarkers for each set of biomarkers from each image of the obtained plurality of images.

3. (canceled)

4. The method of claim 1, wherein the obtaining the at least one set of biomarkers is performed by at least one of a camera or an eye tracker.

5. The method of claim 1, wherein the biomarkers are associated with left and right eyes of the subject.

6. The method of claim 5, wherein the biomarkers include at least one of pupil diameter or pupil area.

7. The method of claim 1, wherein the obtaining the at least one set of biomarkers occurs during the performance of a cognitive task.

8. The method of claim 1, wherein the calculating the score of attention of the subject includes calculating at least one correlation between the biomarkers relating to: 1) the left side of the face over the at least one time period, and, 2) the right side of the face, over the at least one time period.

9. The method of claim 1, additionally comprising: obtaining an overall metric of attention of the subject by combining each said score of attention over the at least one time period.

10. (canceled)

11. The method of claim 8, wherein the overall metric for attention is compared to a threshold in order to diagnose Attention Deficit Disorder (ADD) or Attention Deficit Hyperactivity Disorder (ADHD).

12. (canceled)

13. The method of claim 7, wherein the cognitive task includes presenting to the subject at least one of visual and auditory contents.

14-15. (canceled)

16. A system for monitoring the attention level of a subject, comprising:
an eye tracker for obtaining at least one set of biomarkers from the left side of the face and the right side of the face of the subject during at least one time period; and,
a processor for receiving data associated with the eye tracker, the processor programmed to:
calculate asymmetries between the biomarkers of the at least one set of obtained biomarkers, a score of attention of the subject during the at least one time period.

17. The system of claim 16, wherein the eye tracker includes an imaging apparatus, and wherein the at least one set of biomarkers includes a plurality of sets of biomarkers, and the processor is additionally programmed to:
obtain, from the imaging apparatus, a plurality of images of the face of the subject over the at least one time period; and,
define the biomarkers for each set of biomarkers from each image of the obtained plurality of images.

18. The system of claim 17, wherein the imaging apparatus includes at least one of cameras and eye trackers.

19. The system of claim 17, the eye tracker for obtaining the at least one set of biomarkers includes at least one of an eye tracking device or a camera.

20. The system of claim 16, wherein the processor is additionally programmed to associate the biomarkers with left and right eyes of the subject.

21. The system of claim 20, wherein the biomarkers include at least one of pupil diameter or pupil area.

22. The system of claim 16, wherein the processor is additionally programmed to calculate the score of attention of the subject by calculating at least one correlation between the biomarkers relating to: 1) the left side of the face over the at least one time period; and, 2) the right side of the face, over the at least one time period.

23. The system of claim 22, wherein the processor is additionally programmed to obtain an overall metric of attention of the subject by combining each said score of attention over the at least one time period.

24. (canceled)

25. The system of claim 23, wherein the processor is additionally programmed to compare the overall metric for attention to a threshold in order to diagnose Attention Deficit Disorder (ADD) or Attention Deficit Hyperactivity Disorder (ADHD).

26. (canceled)

27. The system of claim 16, additionally comprising at least one of lights, display or speakers for presenting a cognitive task in at least one of visual or auditory content.

28-29. (canceled)
